(19) United States
(12) Patent Application Publication
(10) Pub. No.: US 2013/0060258 A1
(43) Pub. Date: Mar. 7, 2013

(54) Endoluminal Surgical Instrument for Stapling, Cutting and Dividing

(71) Applicant: Carman Giacomantonio, Halifax (CA)

(72) Inventor: Carman Giacomantonio, Halifax (CA)

(21) Appl. No.: 13/592,089

(22) Filed: Aug. 22, 2012

Related U.S. Application Data
(60) Provisional application No. 61/527,813, filed on Aug. 26, 2011.

(51) Int. Cl. A61B 17/068; A61B 17/3205

(52) U.S. Cl. 606/110; 606/167; 227/180.1

(57) ABSTRACT

A device for cutting, stapling and dividing the bowel allowing surgery to be performed laparoscopically and associated method of use. The device is inserted transanally and positioned within the bowel, ligatures secure the bowel to the device. The bowel is then cut by the device while securing the open end to minimize spillage and the bowel stapled. The bowel can be re-attached using additional staples and further cuts made to allow the device to be removed.

### CROSS REFERENCE TO RELATED APPLICATION(S)

This application claims benefit of U.S. Provisional Application Ser. No. 61/527,813, filed Aug. 26, 2011 the entire disclosure of which is herein incorporated by reference.

### BACKGROUND OF THE INVENTION

1. Field of the Invention

This disclosure relates to a surgical instrument. In particular, the present invention relates to an endoluminal device for use in resection and anastomosis of a portion of the gastrointestinal tract.

2. Description of Related Art

Surgical intervention is typically the treatment of choice for patients with colorectal cancer in all stages, including stage IV. Similarly, inflammatory bowel disease (IBD) such as Crohn's disease and ulcerative colitis may also be treated surgically. Applying traditional surgical techniques, removal of a diseased bowel typically requires a generous laparotomy, or opening of the abdomen, to facilitate resection and anastomosis of the involved segment. Most of the post-operative morbidity experienced by the patient is related to this aspect of the surgery. Reducing situations requiring laparotomy, reduces the complications for patients.

However, colorectal cancers and IBD are often located in a very difficult to reach anatomic positions in the pelvis, presenting surgeons with strategically challenging approaches to their removal. Moreover, instrumentation and technology has not evolved to the point where laparoscopic techniques can be exclusively applied in the pelvis. Consequently, patients requiring surgery for diseases of the rectum, or where a resection and anastomosis will involve the rectum require a standard formal laparotomy for this purpose. These patients experience all of the inherent morbidity associated with that component of their surgery. Thus there is a need for further development of instrumentation for laparoscopic surgery of the rectum.

The following description summarizes the steps involved in surgical removal of a rectum.

To remove a rectum, or a portion of the rectum, a patient is positioned supine with legs separated and supported up in stirrups to facilitate access to the anal orifice. The abdomen is opened using a standard vertical incision in the midline. The rectum sits in the pelvis below the peritoneal reflection (a membrane that separates the abdomen from the pelvis). In preparation of being removed, the rectum must be mobilized by detaching all of the radial attachments of connective tissue and small blood vessels along its length from the side walls of the pelvis down to the floor of the pelvis. Once the disease-bearing portion of the rectum is completely mobilized it needs to be cut and separated from the remaining end piece. This far (distal) end is very difficult to reach with known surgical instruments and devices. A right-angled stapling device is manoeuvred down into the pelvis and the remaining rectum is stapled off and excluded from the proximal diseased portion. Successful completion of this portion of the surgery is impacted by the physical limitations of space and visibility with which to apply and fire the stapler. The rectum is then cut off just above the staple line, while trying not to spill any of the bowel content. Male gender and BMI often limit the extent to which the rectum can be removed. If the surgeon is unable to dissect and staple below the diseased segment because of physical limitations of the patient, the remaining option involves more radical surgery to include complete excision of the rectum plus anus: the so-called abdomino-perineal resection. These patients have far more morbidity including the added burden of a permanent colostomy.

Once the diseased portion of the rectum is removed, the colon may then be re-attached to the remaining portion of the rectum; the so-called rectal stump. This is typically accomplished using an end-to-end stapling device. An end-to-end stapling device essentially has two functional components: a staple deck that is attached to a stapler and inserted into the rectal stump via the anal orifice and an anvil which is sewn into the end of the colon just above (proximal to) the former disease-bearing portion of the rectum. Once positioned and secure in the end of the colon the anvil is then attached to the stapler. The stapler is then closed thereby pressing the deck of staples into the anvil and bending them appropriately to join the ends of the bowel and complete the anastomosis.

### SUMMARY OF THE INVENTION

For these and other reasons known to those of ordinary skill there are described herein devices for cutting, stapling and dividing the bowel allowing surgery to be performed laparoscopically and associated methods of use. A device is inserted transanally and positioned within the bowel, ligatures secure the bowel to the device. The bowel is then cut by the device while securing the open end to minimize spillage and the bowel stapled. The bowel can be re-attached using additional staples and further cuts made to allow the device to be removed.

There is described herein, in an embodiment, an endoluminal device comprising: a substantially cylindrical body including: a head portion having a ligature detent for securing said head portion to a first portion of a bowel; and a base portion releaseably connected to said head portion along an axis of said body and having a ligature detent for securing said base portion to a second portion of said bowel; a first cutting edge directed along said axis; wherein said first cutting edge severs said first portion of said bowel from said second portion of said bowel while said head portion and said base portion are connected to each other; and wherein said head portion and said base portion are detached from each other after said cutting edge has severed said bowel.

In an embodiment, the device further comprises: an anvil portion connectable to said base portion and including a ligature detent for securing said anvil portion to said second portion of said bowel; a staple assembly within said base portion cooperative with said anvil portion for applying a plurality of staples through both said first and said second portions of said bowel to secure said first and said second portions of said bowel together; and a second cutting edge directed along said axis of said body; wherein said second cutting edge severs at least part of said first and said second portions of said bowel from said base portion and said anvil portions to allow said device to be removed from said bowel.

In an embodiment of the device said first cutting edge is a coring knife.

In an embodiment, the device further comprises a control handle releaseably engaged with said base portion, said handle comprising a first activator for activating said first cutting edge, and a second activator for detaching said base portion from said head portion.

In an embodiment, the device further comprises a third ligature detent in said body where said bowel may be secured to said body by a third ligature and said first cutting edge is directed to said third ligature detent severing said bowel on either side of said third ligature.

There is also described herein an endoluminal device for stapling, cutting and dividing a bowel having an open position and a closed position, said device comprising: a base portion, having an axis, a first staple assembly and in communication with a control handle; a circular-symmetric first ligature detent adjacent to and co-axial with said base portion, such that bowel, constrained by a first ligature aligned with said first ligature detent is positioned between said first staple assembly and a first staple anvil; a knife portion adjacent to and co-axial with first ligature detent, having at least one cutting surface retractably extending radially from said axis and said first staple anvil cooperative with said first staple assembly when said device is in a closed position, and a second staple anvil cooperative with a second staple assembly when said device is in a closed position; a circular-symmetric second ligature detent adjacent to and co-axial with said knife portion, such that bowel, constrained by a second ligature aligned with said second ligature detent, is positioned between said second staple assembly and said first staple anvil; and a head portion adjacent to and co-axial with said second ligature detent, having said second staple assembly, releaseably locked with said knife portion; wherein said first and second staple assemblies may be fired to secure said bowel to said base and head portions respectively and said at least one cutting surface extended and rotate to severe said bowel before said head portion is released from said knife portion.

In an embodiment of the device said at least one cutting surface is extended by rotating said control handle in a first direction and retracted by rotating said control handle in a direction opposed to said first direction.

In an embodiment, the device further comprises a control handle releaseably engaged with said base portion, said handle comprising a first activator for activating said cutting edge, and a second activator for releasing said base and head portions.

There is also described herein an endoluminal device for stapling, cutting and dividing a bowel, device comprising: a head portion and a base portion on a common axis, releaseably secured to each other, said base portion in communication with a control handle; a ligature detent between said head portion and said base portion, such that bowel, constrained by a first ligature aligned with said first ligature detent; a clamp detent between said head portion and said base portion encompassing said ligature detent, said clamp detent accepting a clamping ring constraining said bowel between said clamping ring and said head and base portions; a first staple assembly in said base portion cooperative with said clamping ring for placing a plurality of staples through said bowel and securing bowel to said base portion; a first cutting edge associated with said base portion directed along said axis of said body at said clamping ring, whereby said first cutting edge severs said bowel, creating a first portion of said bowel adjacent to said head portion and a second portion of said bowel adjacent to said base portion; wherein said base and head portions may be separated while said second portion of said bowel remains secure to said base portion by said plurality of staples.

In an embodiment of the device said cutting edge is a coring knife.

In an embodiment of the device said clamping ring further comprises a first semicircular clamp joined by a hinge to a second semicircular clamp at said first ends; said first and second semicircular clamps releaseably connected at said second ends.

In an embodiment, the device further comprises: an anvil portion securely receivable by said base portion, said anvil portion secured to first portion of said bowel by one or more ligature detents accepting one or more ligatures securing said bowel to said anvil; a second staple assembly within said base portion cooperative with said anvil portion for applying a plurality of staples through said first and second portions of said bowel securing said first and second portions of said bowel together; a second cutting edge directed along said axis of said body at said anvil portion whereby said second cutting edge severs said bowel at said at least one ligature detent on base portion releasing said first and second portions of said bowel from said base and anvil portions; wherein said device may be removed from said bowel.

There is also described herein a method of removing a portion of a bowel comprising: inserting a device transanally to said portion of said bowel to be removed, said device having a base portion and a head portion releaseably secured together; securing said bowel to said device with at least three ligatures aligned with a second detent on said base portion, a third detent on said head portion and a first detent between said second and third detents; activating a cutting edge in said device to severe said bowel on either side of said first detent into a first portion of said bowel and a second portion of said bowel; releasing said base and head portions of said device from each other; whereby said first portion of said bowel remains secured to said base portion and said second portion remains secured to said head portion.

In an embodiment, the method further comprises: securing said base portion to an anvil portion together; said base portion secured by said first ligature to said first portion of said bowel and said anvil portion secured by a ligature to said second portion of said bowel; activating a staple assembly to secure said first and second portions of said bowel together using a plurality of staples; activating a cutting edge in said base portion to severe said bowel from said base portion and anvil portion; and removing said base and anvil portion transanally; wherein said plurality of staples secure said first and second portions of said bowel together.

### BRIEF DESCRIPTION OF THE DRAWINGS

FIG. 1 is an exploded perspective view of a first embodiment of the invention.

FIG. 2 is a side view of the embodiment of FIG. 1.

FIG. 3 is a front view of the embodiment of FIG. 1.

FIG. 4 is a sectional view taken along line 4-4 of FIG. 2.

FIG. 5 is a perspective view illustration of a rectum with the embodiment of FIG. 1 inserted therein.

FIG. 6 is a side view of the rectum of FIG. 5 with the embodiment of FIG. 1 inserted therein.

FIG. 7 is a sectional view of the rectum with the embodiment of FIG. 1 inserted therein of FIG. 6 taken along line 7-7.

FIG. 8 is a perspective view of a second embodiment of the invention in a closed configuration, entering the bowel.

FIG. 9 is a side view of the embodiment of FIG. 8 in an open configuration within the bowel.

FIG. 10 is a side view of the embodiment of FIG. 9 bound by a first ligature.

FIG. 11 is a side view of the embodiment of FIG. 10 further bound by second and third ligatures.

FIG. 12 is a sectional view of the embodiment of FIG. 11 with inner coring knife extended.

FIG. 13 is a side view of the embodiment of FIG. 12 with a first anvil section of the second embodiment separated from a body section of the embodiment.

FIG. 14 is a side view of the embodiment of FIG. 13 illustrating the introduction of a second anvil section in a new bowel opening to be anastomosed.

FIG. 15 is a sectional view of the embodiment of FIG. 14 wherein the second anvil section is connected to the body section, staples join the rectal stump and the new bowel opening, and an outer coring knife is partially extended.

FIG. 16 is an enlarged view of the embodiment of FIG. 15 wherein the outer coring knife is fully extended.

FIG. 17 is a perspective view of an anastomosis.

FIG. 18 is a perspective exploded view illustration of the embodiment of FIG. 1.

FIG. 19 is a sectional view illustration of the embodiment of FIG. 7.

FIG. 20 is a perspective view of a section of the embodiment of FIG. 19.

FIG. 21A is a perspective view illustration of a third embodiment entering the bowel in a closed configuration.

FIG. 21B is a perspective view illustration of the embodiment of FIG. 21A inserted into the bowel and expanded into an open configuration.

FIG. 21C is a side view illustration of the embodiment of FIG. 21B bound by a first ligature.

FIG. 21D is a perspective view illustration of the embodiment of FIG. 21C showing a two-part magnetic clamping ring.

FIG. 21E is an enlarged sectional view illustration of a portion of the embodiment of FIG. 21D with the magnetic clamping ring applied to the external surface of the bowel in an annular recess of the embodiment with a ring of staples.

FIG. 21F is an enlarged sectional view illustration of a portion of the embodiment of FIG. 21E with an inner coring knife extended.

FIG. 21G is a perspective view illustration of a section of the embodiment of FIG. 21F with the magnetic clamping ring removed.

FIG. 21H is a side sectional view illustration of the embodiment of FIG. 21G with the head portion partially removed from the base portion.

### DESCRIPTION OF THE PREFERRED EMBODIMENT(S)

In embodiments, an endorectal instrument is provided that allows for the stapling, cutting and dividing of the bowel laparoscopically.

### First Embodiment

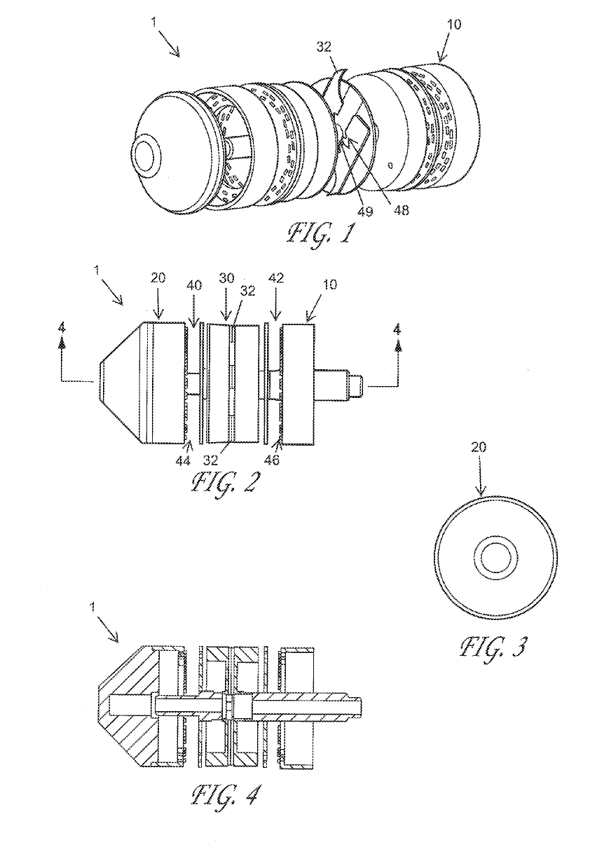

With reference to FIGS. 1, 2 and 18, in a first embodiment, the device 1 is generally cylindrical for inserting transanally into the rectum to the dissection position within the bowel. The outside diameter of the device must be suitable for insertion within the bowel. Preferably the rectum is enclosed within a protective sleeve (not shown) inserted laparoscopically to protect the surrounding structures during the following procedure.

The device 1 has a head portion 20, a knife portion 30 and a base portion 10. When inserted, the base portion 10 is oriented towards the anus. In an open position, two gaps 40, 42 are formed, the first gap 42 between the base portion 10 and the knife portion 30 and the second gap 40 between the knife portion 30 and the head portion 20.

In the first embodiment, the knife portion 30 includes knife blades 32 that in a retracted position are contained within the body of knife portion of the device. The knife blades can be extended from the knife portion as shown in FIG. 1. In the extended portion, the knife blades can be used to cut the bowel.

The head and base portions of the device each contain a staple assembly 44, 46 which apply surgical staples to the bowel to plug the cut portion of the bowel. The staples from the staple assembly may be fired in parallel with the axis of the device 1 into an anvil integrated with the knife portion 30.

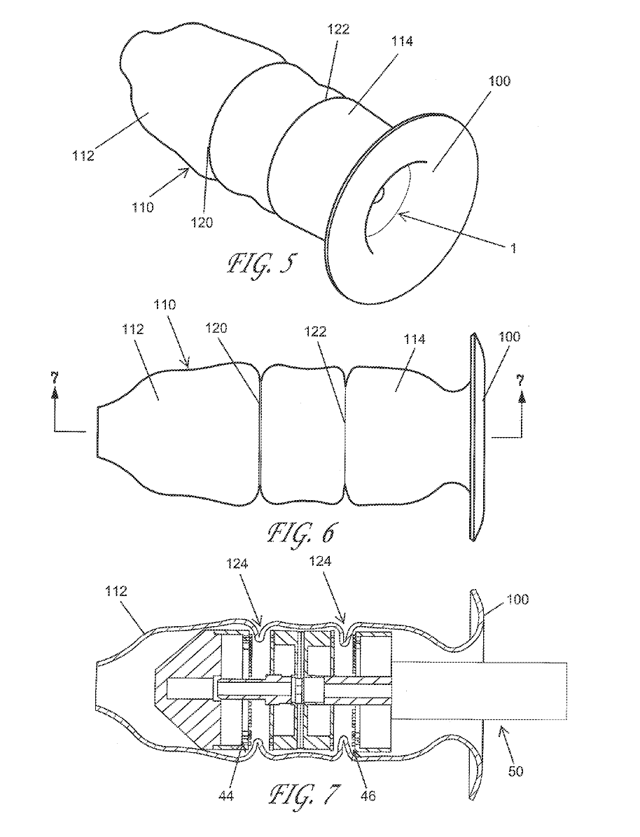

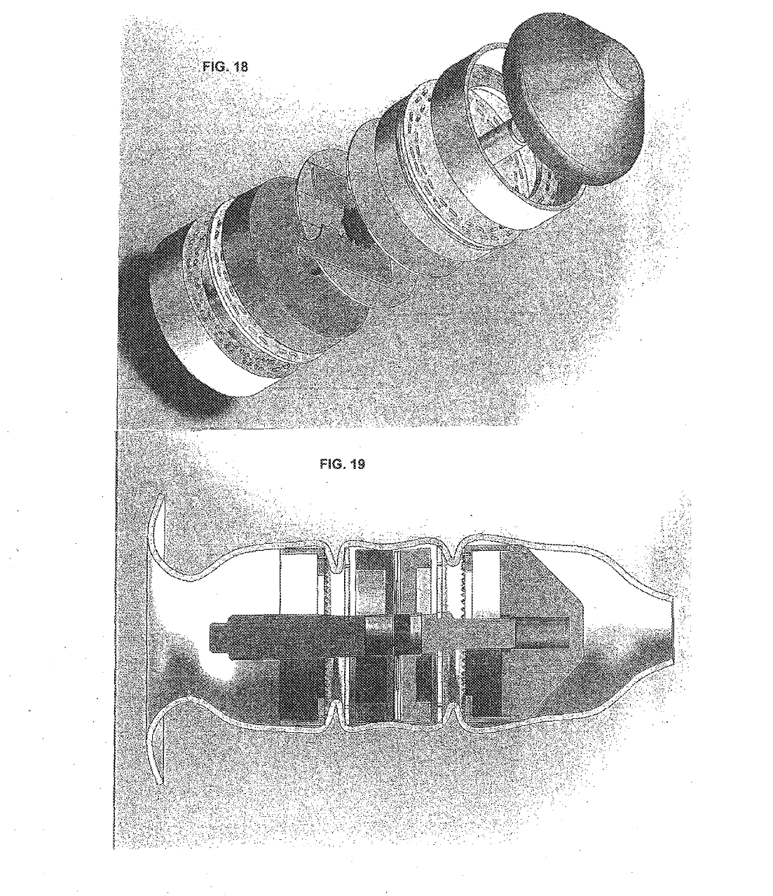

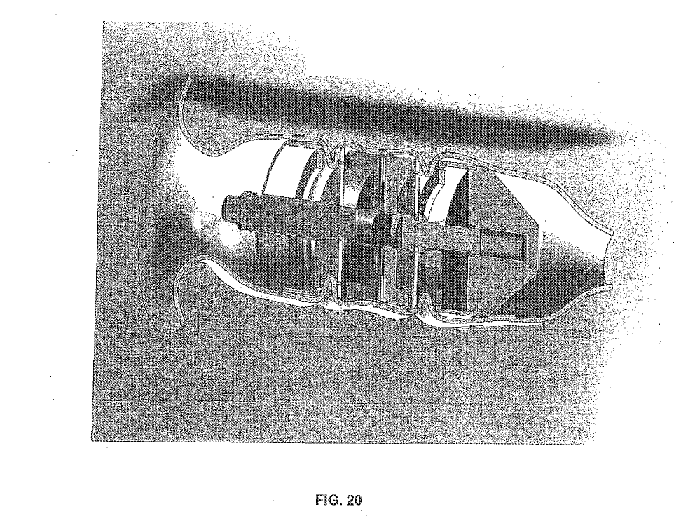

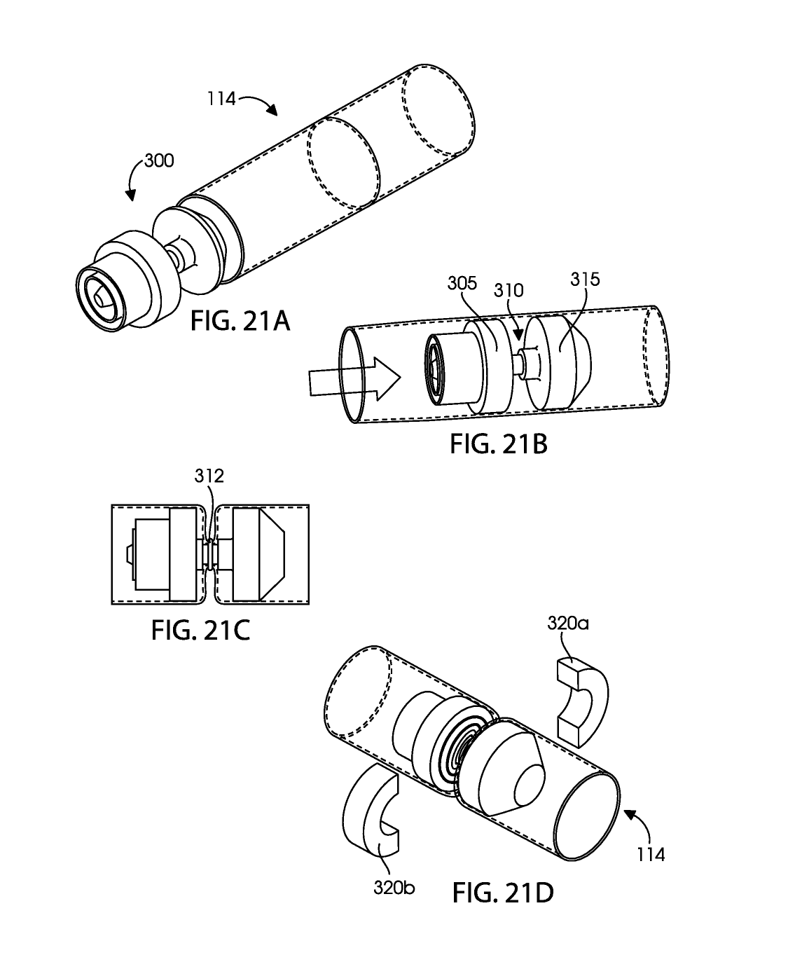

With reference to FIGS. 5, 6, 7, 19 and 20, the device 1 in position within the bowel 110 is shown. The device is inserted from the anus 100 and into the rectum 114 into a position just beyond the section of the bowel that is to be removed.

With the device in the open position in the bowel, ligatures 120 and 122 are placed around the outside of rectum 114 in alignment with the first 42 and second gaps 40 in the device. With the ligatures in place, folds 124 of the bowel are squeezed into the gaps 40, 42. The device is then closed, or tightened using a handle 50 or other means of control so that the gaps are reduced and the folds of the rectum are pinched within the two staple assemblies.

The staple assemblies 44, 46 are fired to place two circular banks of staples in the tissue of the rectum, one above and one below the knife portion of the device.

With the staples in position to hold the rectum, the knives 32 of the knife portion 30 are radially extended from the device 1 and through the wall of the rectum 114. The knives 32 may be extended by rotating the handle 50 of the device, shown in FIG. 7. Rotating the extended knives cuts the wall of the rectum, severing the rectum into two parts. The knives may be rotated by rotating the entire device 1 or the knife portion 30 within the rectum using the handle 50. The knives 32 may be endoknives containing two blades that protrude radially through the wall of the rectum when deployed.

As shown in FIG. 1, the knife blades may include gear tracks 48, or a series of notches on an inner surface that engage with a shaft 49 having teeth. By rotating the shaft in a first direction, the knife blades are extended. Rotating the shaft in a reverse direction retracts the knife blades.

The staple ends serve as caps containing the ends of the rectum to prevent any spillage and to ready the ‘rectal stump’ for reconnection to the colon. The knife blades can then be retraced by counter-rotating the handle 50. By retracting the knife blades, the knife portion can be later removed through the rectum with reduced damage to the surrounding tissue. The device then separates between the knife portion and the head portion so that the severed portion of the rectum, containing the head portion may be removed from the pelvic area. The base portion may remain in the rectal stump.

For re-attaching the rectum, the base portion of the device may form a grommet-like ring that is compatible with an end-to-end stapling device that is used to staple the rectal stump to the portion of the rectum to be reattached.

The device may be made from a durable surgically inert material such as plastic or metal as would be known to a person skilled in the art.

### Second Embodiment

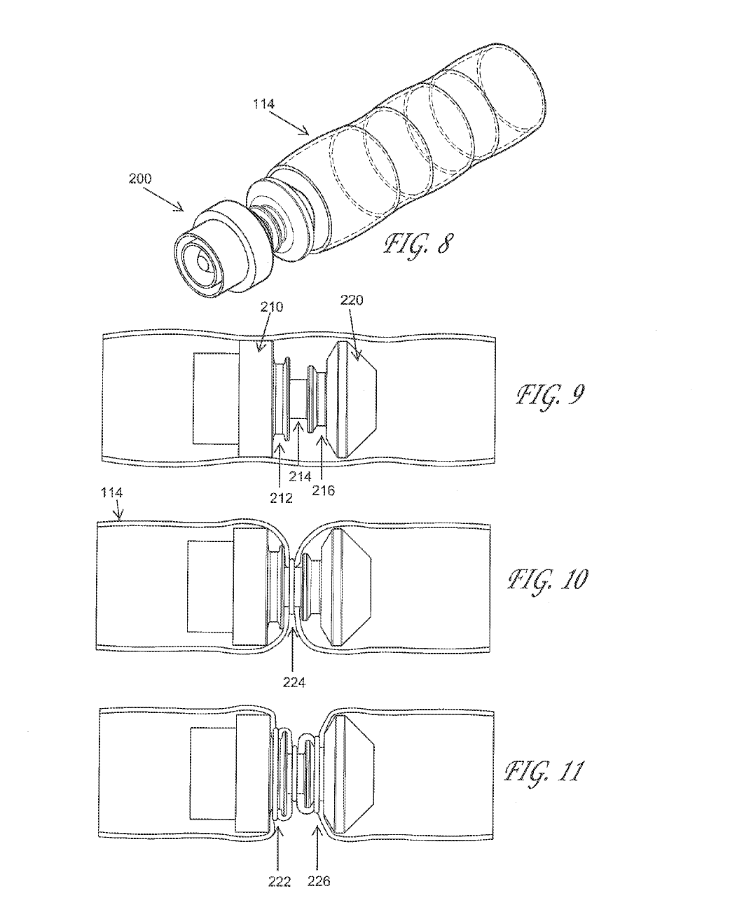

In a second embodiment, the device 200 contains two primary components, a head portion 220 and a base portion 210. As with the first embodiment, the device is inserted into the anus and positioned within the rectum 114 near the portion that is to be removed—see FIG. 8. The device may be inserted into the rectum in a closed position.

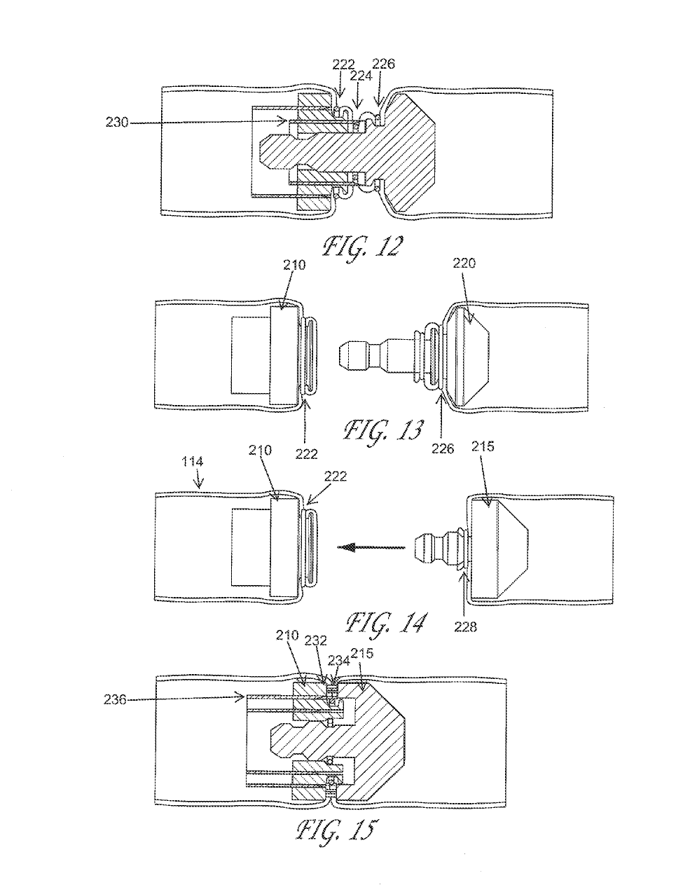

With reference to FIG. 9, the device includes a head portion 220 and a base portion 210. The base portion 210 is oriented towards the anus. Once in position, the device may be manipulated into an open position. In an open position, three ligature detents are exposed 212, 214, and 216.

A first ligature 224 is applied to the rectum in alignment with the middle detent 214 in the device as illustrated in FIG. 10. The ligature 224 secures the bowel 114 around the device 200. Second 222 and third 226 ligatures are applied to the rectum 114 in alignment with the first 212 and third 216 detents as illustrated in FIG. 11. The second and third ligatures are intended to secure the sections that will become the open ends of the bowel from leaking after being cut.

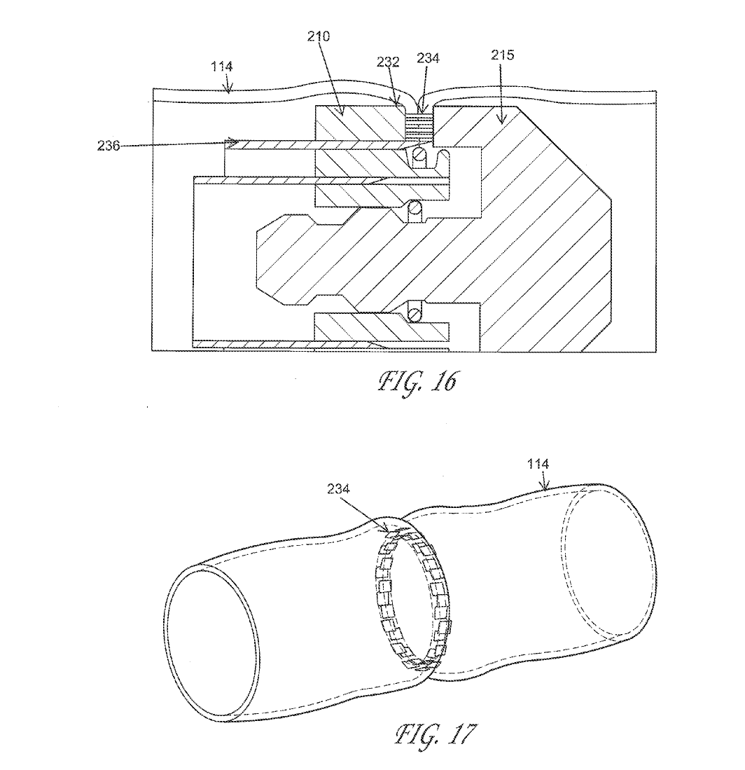

With reference to the cross-section of FIG. 12, the inner coring knife 230 contained within the base portion is fired which cuts the bowel on either side of the first ligature in place around the middle detent. The inner coring knife may be fired using a handle 50 affixed to the device 200, using a mechanical activator such as a rod or wire control linkage or some other activator means such as by electro-mechanical means, wired solenoid, or using a wireless device. The coring knife may be spring loaded so that a release allows the stored spring energy to drive the coring knife through the bowel. The path of the knife may be stopped by an anvil on the head portion of the device between the middle 214 and third detents 216.

The coring knife 230 has a circular cutting surface that cuts the bowel 114 all the way around. The coring knife may be held in place in a sleeve formed in the base portion 210 of the device (not shown).

Once the bowel has been severed as described above, the two portions of the device, the base 210 and head 220 portions may be separated, as illustrated in FIG. 13. In this way, the rectal stump connected to the anus is attached to the base portion 210 by the second ligature 222. The head portion 220 of the device is removed along with a portion of the bowel. While it is being removed, the bowel 114 is secured to the head portion 220 by the third ligature 226 to ensure any spillage from the bowel 114 is minimized.

The base 210 and head 220 portions may be connected using an inserted flanged peg, preferably affixed to the head portion. The base portion may include a clasp that clips over the flanged peg to secure the head portion to the base portion. The handle 50 may be used to release the flanged peg allowing the head portion to be removed from the base portion as described above.

The base and head portions of the device to be detached using a male and female connector as illustrated in FIG. 12 (in cross-section) and FIG. 13. The head portion 220 may include a circulator pin that is received by a corresponding opening in the base portion. In this way, the head and base portions can be secured together for insertion and firing of the inner coring knife.

After a portion of the bowel is removed, an anvil portion 215 of the device 200 is introduced. It is attached to the new section of the bowel 114 by a ligature 228 as indicated in FIG. 14. The anvil portion 215 is mated with the base portion 210 of the device remaining in the rectal stump. The anvil portion 215 may contain a similar male connector as the head portion 220 to mate securely with the same opening in the base portion 210.

Once the anvil 215 and base 210 portions are connected, the staple assembly 232 of the base portion 210 is fired, using the anvil portion 215 as an anvil as indicated in FIG. 15. The staples 234 connect the bowel portions being re-attached. The staple assembly 232 fires surgical staples in a full circular pattern about the axis of the device 200 securing the bowel stump portion to the other portion of the bowel. The staple assembly 232 may be triggered from the device handle 50 or using other means known to a person skilled in the art.

After the staples assembly 232 have fired, the second coring knife 236 is fired which cuts the two ligatures—one on the base portion and one on the anvil portion, as indicated in FIG. 16. The coring knife 236 has a circular cutting surface that cuts the bowel 114 all the way around inside of the staples. The coring knife may be held in place in a sleeve formed in the base portion 210 of the device (not shown).

Once severed, the ligatures 222 and 226 no longer secure the bowel to the device and the device including both the anvil 215 and base 210 portions can then be extracted through the cored opening and out the anus. The portions of the rectum remain with staples holding the sections together as illustrated in FIG. 17. In this way the two portions of the bowel are re-attached end-to-end.

### Third Embodiment

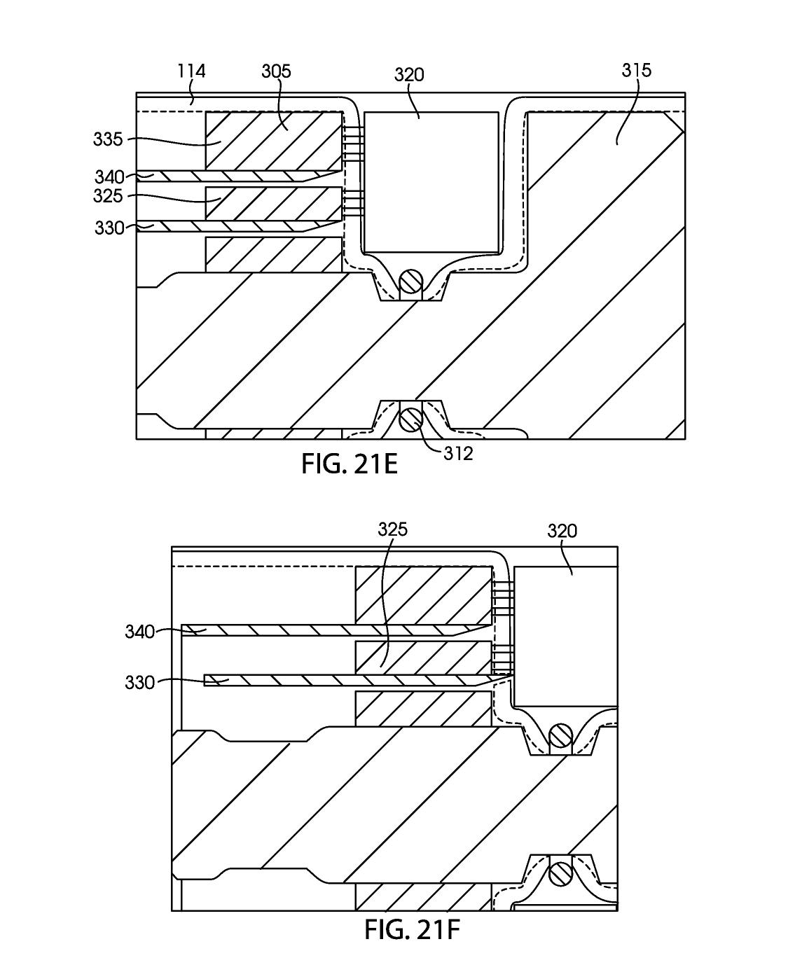

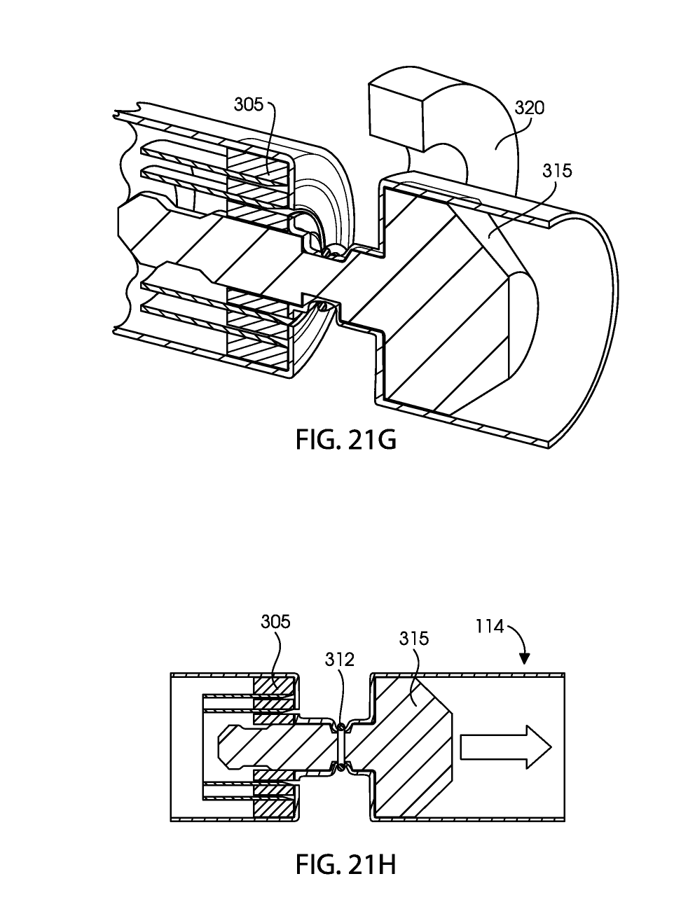

In a third embodiment, an external clamping ring is applied to the device 300 as shown in reference to FIGS. 21A through 21H.

In this embodiment, the device is inserted transanally in a closed position. The device is positioned within the bowel near the area to be removed and expanded into an open position revealing a single detent 310 between a head portion 315 and a base portion 305. A single ligature 312 may be placed around the bowel in alignment with the single detent as illustrated in FIG. 21C. The ligature secures the bowel 114 against the detent 310 of the device 300.

With reference to FIG. 21D, a clamping ring 320 is placed around the bowel 114 and device 300 constraining the bowel 114 against the device 300 at the detent 310. At least a portion of clamping ring 320 may fit between the head 315 and base 305 portions of the device 300 in the detent 310. In this way the clamping device 320 may act as an anvil for stapling assemblies contained within the base portion 305 of the device 300 to be described later.

The clamping ring 320 may be formed in two parts 320a, and, 320b and held together with magnetic force or other securing means. Alternatively, the clamping ring may be a single hinged part, secured by magnetic force or other securing means at a non-hinged point.

With the clamping ring 320 in position between the base 305 and head portions 315, the device is closed upon the clamping ring 320. An inner first staple assembly 325 contained in the base portion is fired against the clamping ring 320, using the clamping ring 320 as an anvil, securing the bowel tissue 114 to the base portion 305 of the device. The inner staple assembly places a ring of staples in the bowel 114 about the axis of the device. Once secure, an inner coring knife 330 is fired against the clamping ring 320 to cut the bowel as illustrated in FIG. 21F. The coring knife has a circular cutting edge about the axis of the device.

Once cut, the clamping ring 320 may be removed and the head 315 and base portions 305 of the device separated as shown in FIGS. 21G and 21H with the ligature 312 on the head portion 315 securing the bowel 114 from inadvertent leakage. The head portion may contain a pin or other means for securing and aligning the head portion with the base portion 305. The base portion may contain a compatible opening for accepting the pin to provide a secure releasable lock on the head portion. As required, portions of the bowel 114 may be removed, such as if there are concerns of a tumour.

The process for re-attaching the bowel is similar to that described earlier for the second embodiment. When re-attaching the bowel, an anvil portion 215 is secured to the new section of the bowel to be re-attached, such as with a ligature 228, similar to that illustrated in FIG. 14.

When the anvil portion 215 is inserted into the base portion 305 and secured, a second staple assembly 335 in the base portion 305 may be fired, securing the bowel tissue of the rectal stump to the tissue of the new section of bowel, similar to that illustrated in FIG. 15.

A second coring knife 340 within the base portion 305 may be applied to sever the first applied staples from the second applied staples. Again, the second coring knife 340 may have a circular cutting surface and may consist of a single circular knife or a plurality of knives forming a predominately circular cutting surface. Once the second coring knife 340 has been applied, the anvil 215 and base portion 305 may be removed through the cored opening leaving the stapled bowel, similar to that illustrated in FIG. 17.

The embodiments are preferably made from moulded plastic, particularly the outer surface of the device. Moving parts, including the gears, control rods and cables and cutting edges may be made from surgical steel or titanium. The materials used to make the device should be surgically acceptable.

The staple assemblies described above may be made using staple deck attached to a stapling device. The staple deck is a pre-loaded cartridge containing the staples. The staples may preferably be made from titanium or other surgically suitable material.

As discussed above, the device is preferably controlled by a control handle 50. The handle preferably includes a fixed handle for manipulation by the surgeon with manipulators. The manipulators are preferably mechanical controls for engaging the coring knives and firing the staple assemblies. The manipulators may use other methods for controlling the operation of the device by the surgeon.

While the device has been described in relation to bowel surgery, it may also be applicable to other types of surgery involving resection and anastomosis of vessels of the body, including, for example the large intestine and esophagus.

While the invention has been disclosed in conjunction with a description of certain embodiments, including those that are currently believed to be the preferred embodiments, the detailed description is intended to be illustrative and should not be understood to limit the scope of the present disclosure. As would be understood by one of ordinary skill in the art, embodiments other than those described in detail herein are encompassed by the present invention. Modifications and variations of the described embodiments may be made without departing from the spirit and scope of the invention.

1. An endoluminal device comprising:
a substantially cylindrical body including:
a head portion having a ligature detent for securing said head portion to a first portion of a bowel; and
a base portion releaseably connected to said head portion along an axis of said body and having a ligature detent for securing said base portion to a second portion of said bowel; and
a first cutting edge directed along said axis;
wherein said first cutting edge severs said first portion of said bowel from said second portion of said bowel while said head portion and said base portion are connected to each other; and
wherein said head portion and said base portion are detached from each other after said cutting edge has severed said bowel.

2. The device of claim 1 further comprising:
an anvil portion connectable to said base portion and including a ligature detent for securing said anvil portion to said second portion of said bowel;
a staple assembly within said base portion cooperative with said anvil portion for applying a plurality of staples through both said first and said second portions of said bowel to secure said first and said second portions of said bowel together; and
a second cutting edge directed along said axis of said body
wherein said second cutting edge severs at least part of said first and said second portions of said bowel from said base portion and said anvil portions to allow said device to be removed from said bowel.

3. The device of claim 1 wherein said first cutting edge is a coring knife.

4. The device of claim 1 further comprising a control handle releaseably engaged with said base portion, said handle comprising a first activator for activating said first cutting edge, and a second activator for detaching said base portion from said head portion.

5. The device of claim 1 further comprising a third ligature detent in said body where said bowel may be secured to said body by a third ligature and said first cutting edge is directed to said third ligature detent severing said bowel on either side of said third ligature.

6. An endoluminal device for stapling, cutting and dividing a bowel having an open position and a closed position, said device comprising:
a base portion, having an axis, a first staple assembly and in communication with a control handle;
a circular-symmetric first ligature detent adjacent to and co-axial with said base portion, such that bowel, constrained by a first ligature aligned with said first ligature detent is positioned between said first staple assembly and a first staple anvil;
a knife portion adjacent to and co-axial with first ligature detent, having at least one cutting surface retractably extending radially from said axis and said first staple anvil cooperative with said first staple assembly when said device is in a closed position, and a second staple anvil cooperative with a second staple assembly when said device is in a closed position;
a circular-symmetric second ligature detent adjacent to and co-axial with said knife portion, such that bowel, constrained by a second ligature aligned with said second ligature detent, is positioned between said second staple assembly and said first staple anvil; and
a head portion adjacent to and co-axial with said second ligature detent, having said second staple assembly, releaseably locked with said knife portion;
wherein said first and second staple assemblies may be fired to secure said bowel to said base and head portions respectively and said at least one cutting surface extended and rotate to severe said bowel before said head portion is released from said knife portion.

7. The device of claim 6 wherein said at least one cutting surface is extended by rotating said control handle in a first direction and retracted by rotating said control handle in a direction opposed to said first direction.

8. The device of claim 6 further comprising a control handle releaseably engaged with said base portion, said handle comprising a first activator for activating said cutting edge, and a second activator for releasing said base and head portions.

9. An endoluminal device for stapling, cutting and dividing a bowel, device comprising:
a head portion and a base portion on a common axis, releaseably secured to each other, said base portion in communication with a control handle;
a ligature detent between said head portion and said base portion, such that bowel, constrained by a first ligature aligned with said first ligature detent;
a clamp detent between said head portion and said base portion encompassing said ligature detent, said clamp detent accepting a clamping ring constraining said bowel between said clamping ring and said head and base portions;
a first staple assembly in said base portion cooperative with said clamping ring for placing a plurality of staples through said bowel and securing bowel to said base portion; and
a first cutting edge associated with said base portion directed along said axis of said body at said clamping ring, whereby said first cutting edge severs said bowel, creating a first portion of said bowel adjacent to said head portion and a second portion of said bowel adjacent to said base portion;
wherein said base and head portions may be separated while said second portion of said bowel remains secure to said base portion by said plurality of staples.

10. The device of claim 9 wherein said cutting edge is a coring knife.

11. The device of claim 9 wherein said clamping ring further comprises a first semicircular clamp joined by a hinge to a second semicircular clamp at said first ends; and said first and second semicircular clamps releaseably connected at said second ends.

12. The device of claim 9 further comprising:
an anvil portion securely receivable by said base portion, said anvil portion secured to first portion of said bowel by one or more ligature detents accepting one or more ligatures securing said bowel to said anvil;
a second staple assembly within said base portion cooperative with said anvil portion for applying a plurality of staples through said first and second portions of said bowel securing said first and second portions of said bowel together; and
a second cutting edge directed along said axis of said body at said anvil portion whereby said second cutting edge severs said bowel at said at least one ligature detent on base portion releasing said first and second portions of said bowel from said base and anvil portions;
wherein said device may be removed from said bowel.

13. A method of removing a portion of a bowel comprising:
inserting a device transanally to said portion of said bowel to be removed, said device having a base portion and a head portion releaseably secured together;
securing said bowel to said device with at least three ligatures aligned with a second detent on said base portion, a third detent on said head portion and a first detent between said second and third detents;
activating a cutting edge in said device to severe said bowel on either side of said first detent into a first portion of said bowel and a second portion of said bowel; and
releasing said base and head portions of said device from each other; whereby said first portion of said bowel remains secured to said base portion and said second portion remains secured to said head portion.

14. The method of claim 13 further comprising:
securing said base portion to an anvil portion together; said base portion secured by said first ligature to said first portion of said bowel and said anvil portion secured by a ligature to said second portion of said bowel;
activating a staple assembly to secure said first and second portions of said bowel together using a plurality of staples;
activating a cutting edge in said base portion to severe said bowel from said base portion and anvil portion; and
removing said base and anvil portion transanally;
wherein said plurality of staples secure said first and second portions of said bowel together.
